(19) United States
(12) Patent Application Publication
(10) Pub. No.: US 2016/0166138 A1
(43) Pub. Date: Jun. 16, 2016

(54) IMAGE PICK-UP WINDOW DEFOGGING FUNCTION-EQUIPPED BUILT-IN CAMERA HAND PIECE

(71) Applicant: THE YOSHIDA DENTAL MFG. CO., LTD., Tokyo (JP)

(72) Inventors: Michizo YAMANAKA, Tokyo (JP); Takeshi HASEGAWA, Tokyo (JP)

(73) Assignee: THE YOSHIDA DENTAL MFG. CO., LTD., Tokyo (JP)

(21) Appl. No.: 14/909,665

(22) PCT Filed: Aug. 1, 2014
(86) PCT No.: PCT/JP2014/070316

§ 371 Date: Feb. 2, 2016

(30) Foreign Application Priority Data
Aug. 2, 2013 (JP) 2013-161418

(51) Int. Cl. A61B 1/253; A61B 1/05

(52) U.S. Cl. CPC A61B 1/253; A61B 1/05

(57) ABSTRACT

The present invention provides an image pick-up window defogging function-equipped built-in camera hand piece having a function of treating an affected area and an image observation function of observing the affected area or the like with a camera and capable of defogging the outer surface of an image pick-up window by preventing adhesion of cutting waste and water droplets to the image pick-up window. An image pick-up window defogging function-equipped built-in camera hand piece 1 of the present invention is provided with a defogging air flow path mechanism 4 in a surrounding position of an image pick-up window 15 arranged facing a cutting site of a cutting tool 16, wherein the defogging air flow path mechanism 4 branches from the air flow path system 91 and injects an air flow within a region from the outer surface of the image pick-up window 15 to the cutting site of the cutting tool 16 to shield an area between the outer surface of the image pick-up window 15 and the cutting site by the air flow to defog the outer surface of the image pick-up window 15.

### TECHNICAL FIELD

The present invention relates to an image pick-up window defogging function-equipped built-in camera hand piece and more particularly to an image pick-up window defogging function-equipped built-in camera hand piece having a function of treating an affected area, and a function of observing the affected area and surrounding region thereof by using a camera image and capable of defogging the image pick-up window for the camera.

### BACKGROUND ART

In the field of dental treatment, a practitioner treats an affected area (treatment site) such as a carious tooth or the like in the oral cavity by frequently using a cutting hand piece for cutting and removing a dentine part of the affected area while visually observing the treatment site.

Furthermore, in recent years, there has been developed a dental hand piece having an image photographing function for intraoral observation in addition to the treatment function.

For example, Patent Document 1 proposes a dental hand piece device having an observation function, the dental hand piece device including: an observation aperture serving as illumination in the surroundings of a tool mounting part of a head part of a dental hand piece; a video camera head provided in the inside of the dental hand piece; an illumination lamp; an observation optical fiber for transmitting a video image captured from the observation aperture serving as illumination so that the video image is focused and formed on the front surface of the video camera head; an illumination optical fiber; and a video signal cable for transmitting the video signal from the video camera head to a video control unit.

In the case of the dental hand piece device in Patent Document 1, however, cutting waste or the like generated during treatment of the affected area with a cutting tool adheres to the observation aperture in the surroundings of the tool mounting part, while the dental hand piece device does not have a defogging function for preventing the fogging of the observation aperture, though having the function of treating an affected area and the function of observing the affected area and the surrounding region thereof by using a video image, thus having a problem that it is difficult to obtain a clear video image of the affected area and the surrounding region thereof by using a video camera.

### CITATION LIST

### Patent Documents

Patent Document 1; Japanese Patent Application Laid-Open No. Hei 9-56730

### SUMMARY OF THE INVENTION

### Problems to be Solved by the Invention

A problem to be solved by the present invention is that there is no image pick-up window defogging function-equipped built-in camera hand piece having a function of treating an affected area and an image observation function of observing the affected area and the surrounding region thereof by using a camera and capable of preventing the adhesion of cutting waste, water droplets, and the like to an image pick-up window during treatment of the affected area with a cutting tool so as to defog the outer surface of the image pick-up window.

### Means for Solving the Problems

The image pick-up window defogging function-equipped built-in camera hand piece according to the present invention is most primarily characterized by including: a hand piece body equipped with a head part on the distal end side with a cutting tool for treating an affected area detachably mounted on the head part; an air flow path system for guiding compressed air; a water flow path system for guiding pressurized water for a water flow injected toward the cutting tool; an image pick-up window for the camera provided in a surrounding region of a position where the cutting tool on the head part is mounted; a built-in camera for picking up affected area, image information incident through the image pick-up window; and an image pick-up optical system, arranged in a range from the inside of the image pick-up window to the built-in camera, wherein a defogging air flow path mechanism is provided in a surrounding position of the image pick-up window, the defogging air flow path mechanism branching from the air flow path system and injecting an air flow within a region from the outer surface of the image pick-up window to a cutting site of the cutting tool to shield an area between the outer surface of the image pick-up window and the cutting site by the air flow to defog the outer surface of the image pick-up window.

### Advantageous Effect of the Invention

According to the present invention of claim 1, in a hand piece equipped with a head part on the distal end side with a cutting tool for treating an affected area detachably mounted on the head part like a hand piece such as an air turbine, an ultrasonic scaler, or the like, an air flow from the defogging air flow path mechanism prevents the adhesion of cutting waste or water droplets generated during a cutting work in the affected area to the outer surface of the image pick-up window to defog the image pick-up window, thereby enabling the provision of an image pick-up window defogging function-equipped built-in camera hand piece capable of picking up a clear color image by guiding light from the image pick-up window through an image pick-up optical system to the camera.

According to the present invention of claim 2, in a hand piece including: a hand piece body having a grip part equipped with a head part on the distal end side with a cutting tool for treating an affected area detachably mounted on the head part and having one coupling part at the rear end of the grip part; and a dental tube part having the other coupling part detachably mounted on the hand piece body, like a hand piece such as an air turbine, an air motor, a micromotor, or the like, an air flow from the defogging air flow path mechanism prevents the adhesion of cutting waste or water droplets generated during a cutting work in the affected area to the outer surface of the image pick-up window to defog the image pick-up window, thereby enabling the provision of an image pick-up window defogging function-equipped built-in camera hand piece capable of picking up a clear color image by guiding light from the image pick-up window through the image pick-up optical system to the camera.

According to the present invention of claim 3, in an air turbine hand piece, an air flow from the defogging air flow path mechanism prevents the adhesion of cutting waste or water droplets generated during a cutting work in the affected area to the outer surface of the image pick-up window to defog the image pick-up window, thereby enabling the provision of an image pick-up window defogging function-equipped. built-in camera hand piece capable of picking up a clear color image by guiding light from the image pick-up window through the image pick-up optical system to the camera.

According to the present invention of claim 4, with a configuration in which the built-in camera for picking up an image of the affected area is provided in the distal end portion of the coupling part located inside the hand piece body, an air flow from the defogging air flow path mechanism prevents the adhesion of cutting waste or water droplets generated during a cutting work in the affected area to the outer surface of the image pick-up window to defog the image pick-up window, thereby enabling the provision of an image pick-up window defogging function-equipped built-in camera hand piece capable of picking up a clear color image by guiding light from the image pick-up window through the image pick-up optical system to the camera.

According to the present invention of claim 5, with a configuration in which the built-in camera is provided inside the image pick-up window of the hand piece body, an air flow from the defogging air flow path mechanism prevents the adhesion of cutting waste or water droplets generated during a cutting work in the affected area to the outer surface of the image pick-up window to defog the image pick-up window, thereby enabling the provision of an image pick-up window defogging function-equipped built-in camera hand piece capable of picking up a clear color image by guiding light from the image pick-up window through the image pick-up optical system to the camera.

According to the present invention of claim 6, with a configuration in which the built-in camera is provided in the distal end portion of the coupling part of the dental tube part, an air flow from the defogging air flow path mechanism prevents the adhesion of cutting waste or water droplets generated during a cutting work in the affected area to the outer surface of the image pick-up window to defog the image pick-up window, thereby enabling the provision of an image pick-up window defogging function-equipped built-in camera hand piece capable of picking up a clear color image by guiding light from the image pick-up window through the image pick-up optical system to the camera.

According to the present invention of claim 7, in the image pick-up window defogging function-equipped built-in camera hand piece described in any one of claims 1 to 6, the defogging air flow path mechanism provided therein blows an air flow from a defogging air flow path, which branches from the air flow path system for guiding compressed air to the head part, toward the outer surface of the image pick-up window to defog the image pick-up window. With this configuration, it is possible to provide an image pick-up window defogging function-equipped built-in camera hand piece capable of picking up a clear color image by preventing the adhesion of cutting waste or water droplets generated during a cutting work in the affected area to the outer surface of the image pick-up window to defog the image pick-up window and guiding light from the image pick-up window through the image pick-up optical system to the camera.

According to the present invention of claim 8, in the image pick-up window defogging function-equipped built-in camera hand piece described in any one of claims 1 to 6, the defogging air flow path mechanism provided therein injects an air flow from a defogging air flow path, which branches from the air flow path system for guiding the compressed air and reaching a position around the outer surface portion of the image pick-up window, in a conical form in the forward direction of the image pick-up window to form an air curtain to defog the image pick-up window. With this configuration, it is possible to provide an image pick-up window defogging function-equipped built-in camera hand piece capable of picking up a clear color image by preventing the adhesion of cutting waste or water droplets generated during a cutting work in the affected area to the outer surface of the image pick-up window to defog the image pick-up window and guiding light from the image pick-up window through the image pick-up optical system to the camera.

According to the present invention of claim 9, in the image pick-up window defogging function-equipped, built-in camera hand piece described in any one of claims 1 to 6, the defogging air flow path mechanism provided therein injects an air flow straight from a defogging air flow path, which branches from the air flow path system for guiding the compressed air to the head part, toward a space region between the outer surface of the image pick-up window and the cutting tool to form an air curtain to defog the image pick-up window. With this configuration, it is possible to provide an image pick-up window defogging function-equipped built-in camera hand piece capable of picking up a clear color image by preventing the adhesion of cutting waste or water droplets generated during a cutting work in the affected area to the outer surface of the image pick-up window to defog the image pick-up window and guiding light from the image pick-up window through the image pick-up optical system to the camera.

As described above, the defogging function in the present invention includes not only a function of preventing water vapor from adhering to the outer surface of the image pick-up window, but also a function of preventing the adhesion of water droplets, cutting waste, and the like.

### BRIEF DESCRIPTION OF THE DRAWINGS p FIG. 1 is a partially cutaway front view of a built-in camera hand piece according to a first embodiment of the present invention.

FIG. 2 is a partially cutaway schematic explanatory diagram illustrating that a coupling part on the dental tube part side is attachable/detachable to/from a coupling part on the grip part side in the built-in camera hand piece according to the first embodiment.

FIG. 3 is a structure explanatory diagram illustrating a state where the coupling part on the grip part side is detached from the coupling part on the dental tube part side in the built-in camera hand piece according to the first embodiment.

FIG. 4 is a partially cutaway schematic enlarged view of a hand piece body including a head part in the built-in camera hand piece according to the first embodiment.

FIG. 5 is an enlarged explanatory diagram of a rod fiber according to the first embodiment.

FIG. 6 is a schematic sectional view illustrating a color camera module in the first embodiment.

FIG. 7 is a schematic side view illustrating the color camera module in the first embodiment.

FIG. 8 is a schematic side view illustrating a camera unit in the first embodiment.

FIG. 9 is a schematic sectional view taken along line A-A of FIG. 8.

FIG. 10 is an explanatory diagram illustrating an optical arrangement relation between a camera unit, a rod fiber, and an objective lens in the first embodiment.

FIG. 11 is a circuit diagram illustrating a driving circuit of light emitting elements in the first embodiment.

FIG. 12 is a block diagram illustrating a schematic configuration of a drive control unit in the first embodiment.

FIG. 13 is a diagram illustrating an example of a color image display obtained by a color image display unit in the first embodiment.

FIG. 14 is a partially cutaway schematic enlarged view of a hand piece body including a head part in a variation of the built-in camera hand piece according to the first embodiment.

FIG. 15 is a schematic structure diagram of a defogging air flow path mechanism viewed from the image pick-up window side in the built-in camera hand piece according to the first embodiment.

FIG. 16 is a partially enlarged schematic sectional view in another variation of the built-in camera hand piece according to the first embodiment.

FIG. 17 is a schematic block diagram of a built-in camera hand piece according to a second embodiment of the present invention.

FIG. 18 is a partially cutaway schematic enlarged view of a hand piece body including a head part of the built-in camera hand piece according to the second embodiment.

FIG. 19 is a partially cutaway front view of a built-in camera hand piece according to a third embodiment of the present invention.

FIG. 20 is a structure explanatory diagram illustrating a state where a coupling part on the grip part side is detached from a coupling part on the dental tube part side in the built-in camera hand piece according to the third embodiment.

### MODE FOR CARRYING OUT THE INVENTION

The present invention achieves an object of providing an image pick-up window defogging function-equipped built-in camera hand piece having a function of treating an affected area and an image observation function of observing the affected area and the surrounding region thereof with a camera and capable of preventing the adhesion of cutting waste, water droplets, and the like to the image pick-up window during treatment of the affected area with a cutting tool and defogging the outer surface of the image pick-up window by means of a configuration including: a hand piece body having a grip part equipped with a head part on the distal end side with the cutting tool for treating the affected area detachably mounted on the head part and having one coupling part at the rear end of the grip part; a dental tube part having the other coupling part detachably mounted on the hand piece body; an air flow path system for guiding compressed air for an air flow injected from the head part through the dental tube part toward the cutting tool; a water flow path system for guiding pressurized water for a water flow injected from the head part toward the cutting tool, the pressurized water being pressure-fed to the head part of the hand piece body through the dental tube part; an image pick-up window for the camera provided in a surrounding region of a position where the cutting tool on the head part is mounted; a built-in camera for picking up affected area image information incident through the image pick-up window; and an image pick-up optical system arranged in a range from the inside of the image pick-up window to the built-in camera, wherein a defogging air flow path mechanism is provided in a surrounding position of the image pick-up window, the defogging air flow path mechanism branching from the air flow path system and injecting an air flow within a region from the outer surface of the image pick-up window to a cutting site of the cutting tool to shield an area between the outer surface of the image pick-up window and the cutting site by the air flow to defog the outer surface of the image pick-up window.

### EMBODIMENTS

Hereinafter, an image pick-up window defogging function-equipped built-in camera hand piece (hereinafter, referred to as “built-in camera hand piece”) according to embodiments of the present invention will be described with reference to accompanying drawings.

### First Embodiment

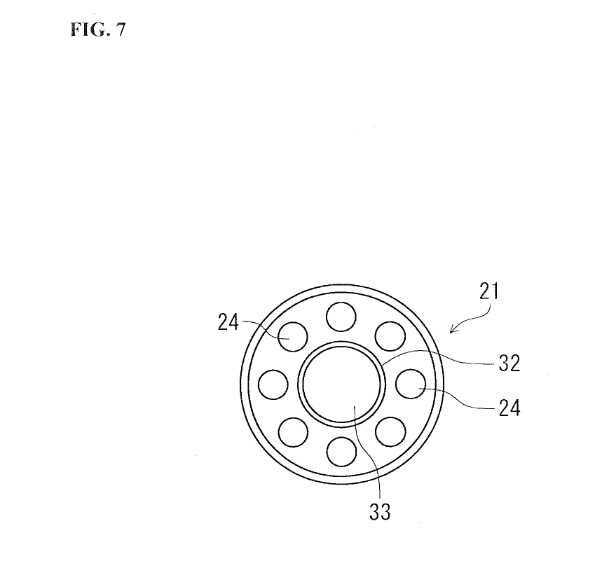

A built-in camera hand piece 1 of the first embodiment will be described with reference to FIGS. 1 to 16.

The built-in camera hand piece 1 of the first embodiment is configured to be of, for example, an air-turbine-driven type as illustrated in FIGS. 1 to 4 and includes: a hand piece body 2 equipped with a head part 14, on which a cutting tool 16 for treating an affected area P is detachably mounted, on the distal end side and including a grip part 3 equipped with a coupling part 11a inside on the rear end side; and a dental tube part 12 equipped with a coupling part 11b attachable/detachable to/from the coupling part 11a of the grip part 3. The coupling part 11a (on the hand piece body 2 side) and the coupling part lib (on the dental tube part 12 side) constitute the coupling part 11.

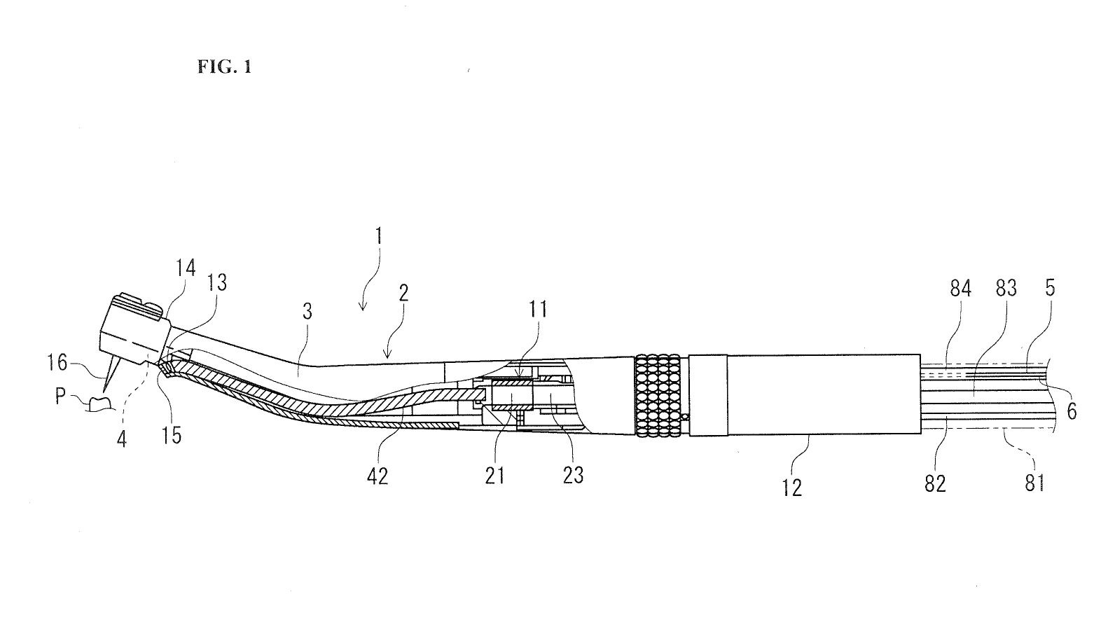

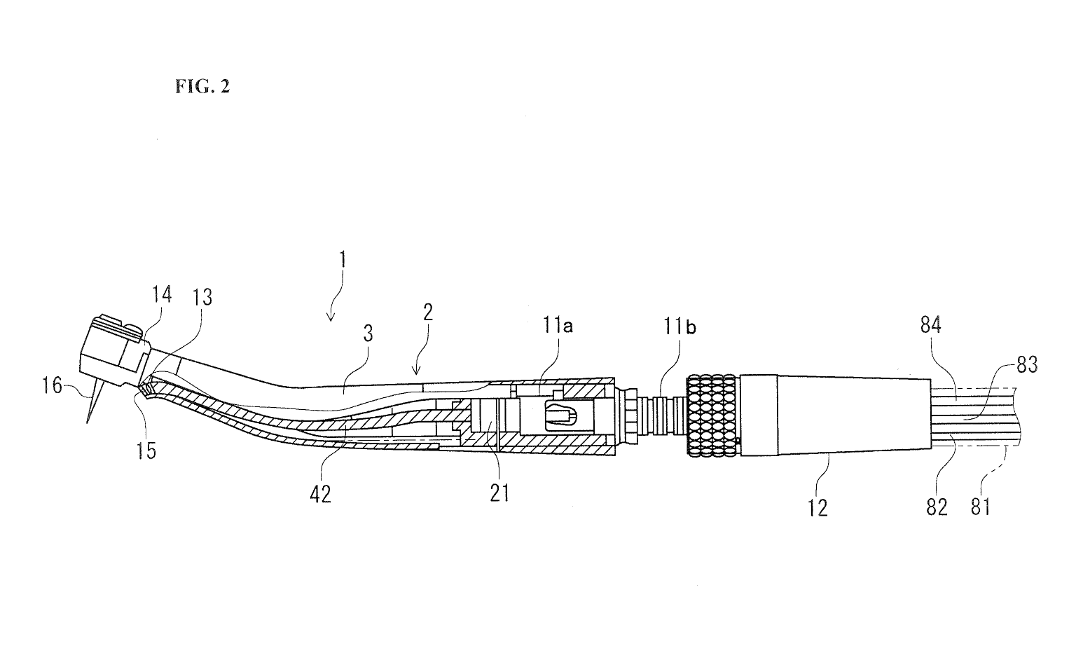

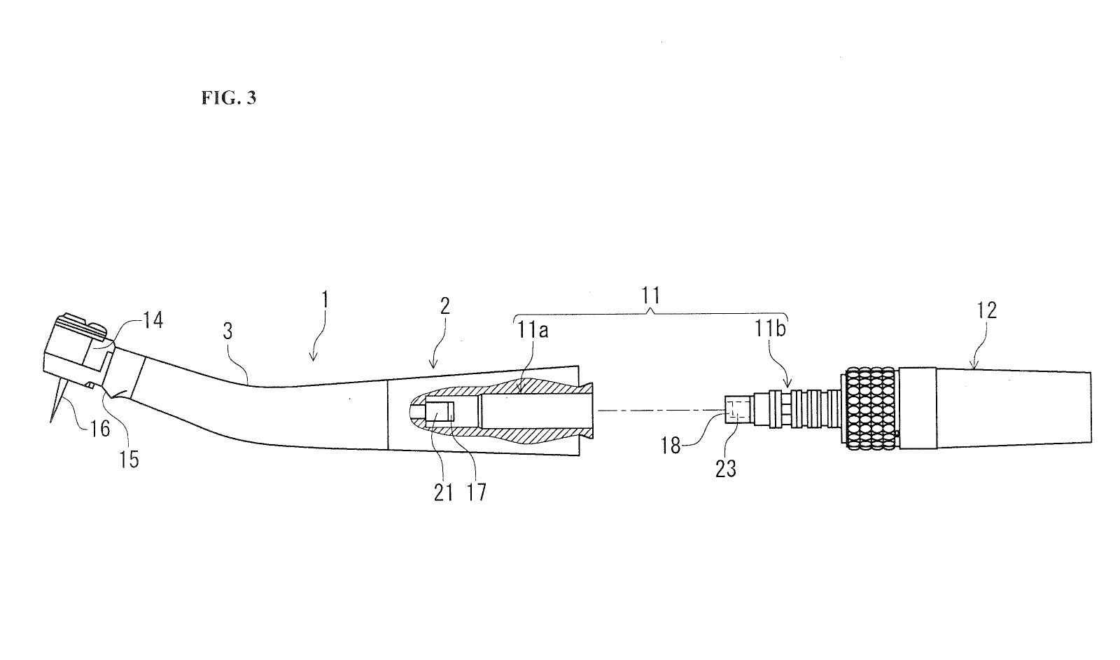

In the built-in camera hand piece 1, the coupling parts 11a and 11b are configured to be rotatably and detachably coupled to each other in a coaxial arrangement by bearing coupling as illustrated in FIGS. 2 and 3.

The dental tube part 12 has a connection hose 81 connected to a dental treatment unit, which is not illustrated.

The connection hose 81 contains an air pipe 82 constituting an air flow path system 91 for guiding a compressed air supplied from a dental treatment unit not illustrated, a water pipe 83 constituting a water flow path system 92 for guiding pressurized water, and an electric cable 84 including a signal output cable 5 and a light emitting element driving cable 6.

Furthermore, in the grip part 3 including the dental tube part 12, the coupling part 11, and the head part 14, there are provided: an air flow path system 91 for guiding compressed, air for an air flow for an air turbine 90 pressure-fed to the head part 14 of the hand piece body 2 through the dental tube part 12 and for an air flow injected from the head part 14 toward the cutting tool 16; and a water flow path system 92 for guiding pressurized water for a water flow pressure-fed to the head part 14 of the hand piece body 2 through the dental tube part 12 and injected from the head part 14 toward the cutting tool 16.

Detailed description is omitted here with respect to a driving flow path system of the air turbine 90 for rotating the cutting tool 16 mounted on the head part 14.

The built-in camera hand piece 1 houses a color camera module 21 which is a camera for picking up an image of an affected area P in the oral cavity in the inside of the hand piece body 2 and in the central portion on the distal end side of the coupling part 11a as illustrated in FIGS. 1 to 3.

Furthermore, an image pick-up window 15, which is formed of, for example, transparent glass material or transparent synthetic resin material, for a color camera module 21 is provided toward the cutting site of the cutting tool 16 in the position near the head part 14 in the grip part 3, and an image pick-up optical system 41 is arranged with the light incident end facing the image pick-up window 15 and with the light emitting end facing the color camera module 21 in the inside of the hand piece body 2.

The image pick-up optical system 41 includes an objective lens 13 arranged in the inside of the image pick-up window 15 and a rod fiber 42 arranged with the incident end facing the objective lens 13 at a predetermined interval and with the emitting end facing a condenser lens unit 33 constituting the color camera module 21 at a predetermined interval.

Moreover, as illustrated in FIG. 3, the output end of the color camera module 21 is connected to a camera cable 23 through a connector 17 in the coupling part 11a and a connector 18 in the coupling part 11b on the dental tube part 12 side.

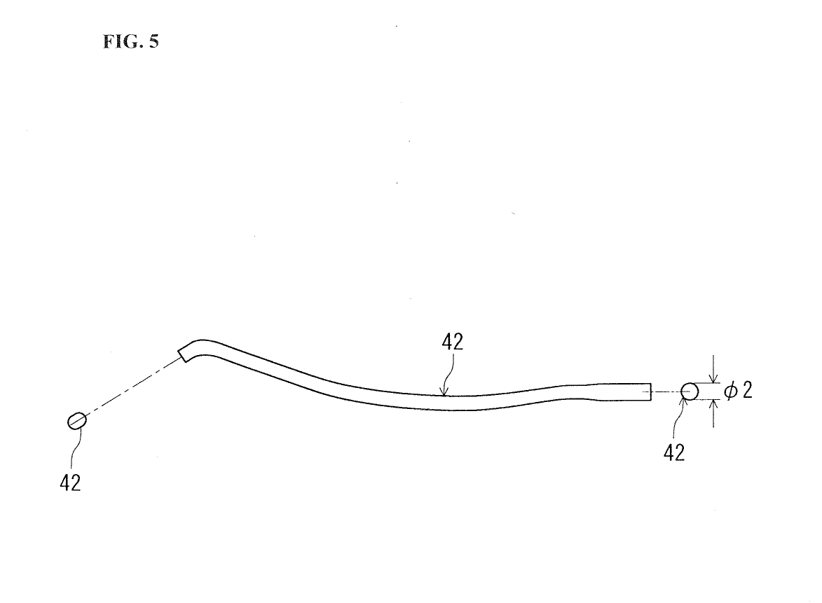

As illustrated in FIG. 5, multi-component glass is used for all of the core, cladding, and skin tube of the rod fiber 42 and the rod fiber 42 has a step index type structure that provides different refractive indices in stages and has optical characteristics of a light receiving angle of about 70 degrees and a numerical aperture (NA) of 0.57.

Moreover, in the rod fiber 42, the shape of the light emitting end thereof has, for example, a fiber diameter Φ2 of about 2.4 mm, the light incident end has an oval shape, for example, with a major axis of about 2.85 mm and a minor axis of about 2.00 mm,

Furthermore, the rod fiber 42 has a specification with autoclave resistance of within 90% with respect to an initial transmittance after 350 cycles under the autoclave conditions of 135° C., 100% RH, and 3 minutes.

The built-in camera hand, piece 1 incorporates a defogging air flow path mechanism 4, which is formed by branching from the air flow path system 91 and injects an air flow into the region between the tooth tip of the cutting tool 16 and the outer surface of the image pick-up window 15 to shield an area between the outer surface of the image pick-up window 15 and the cutting site by means of the air flow in order to defog the outer surface of the image pick-up window 15.

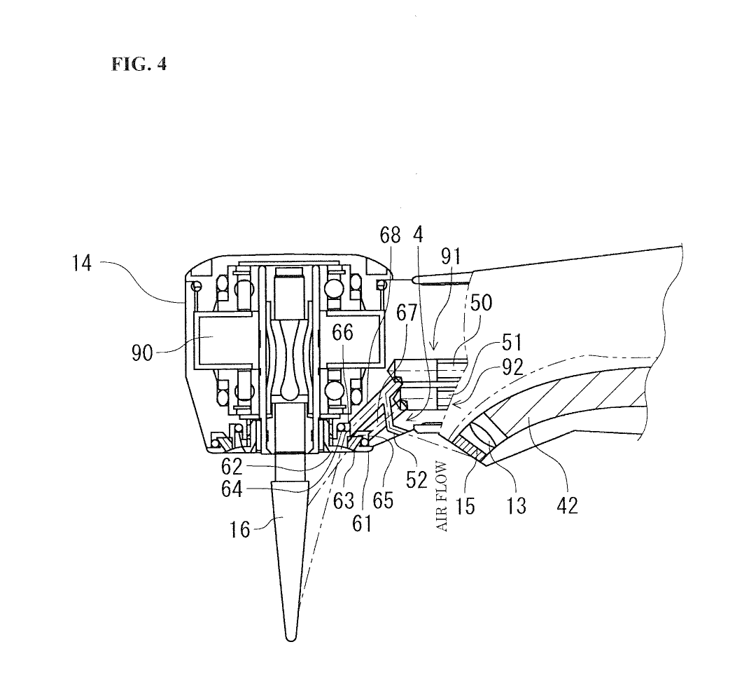

The following describes the defogging air flow path mechanism 4 in detail with reference to FIG. 4.

In the internal region of the grip part 3 of the hand piece body 2 and that of the head part 14, there are provided a water flow path 51 constituting the water flow path system 92 and an air flow path 50 constituting the air flow path system 91, a ring 61 is fixed to the undersurface of the head part 14 through a nut 62 in the position near the location where the cutting tool 16 is attached, a water injection hole 63 is formed in the outside of the undersurface of the ring 61, and an air injection hole 64 is provided in the inside adjacent to the water injection hole 63.

In addition, the water injection hole 63 is connected to the water flow path 51 via a peripheral groove 65 formed on the head part 14 side and a connecting pipeline 67, and the air injection hole 64 is connected to the air flow path 50 via a peripheral groove 66 and a connecting pipeline 68.

Furthermore, there is provided a defogging air flow path 52 constituting the defogging air flow path mechanism 4, which is branched on the way from the air flow path 50 and is bent toward the image pick-up window 15. An air flow is then blown from the defogging air flow path 52 toward the outer surface of the image pick-up window 15, so that the air flow shields the area between the outer surface of the image pick-up window 15 and the cutting site, thereby defogging the outer surface of the image pick-up window 15.

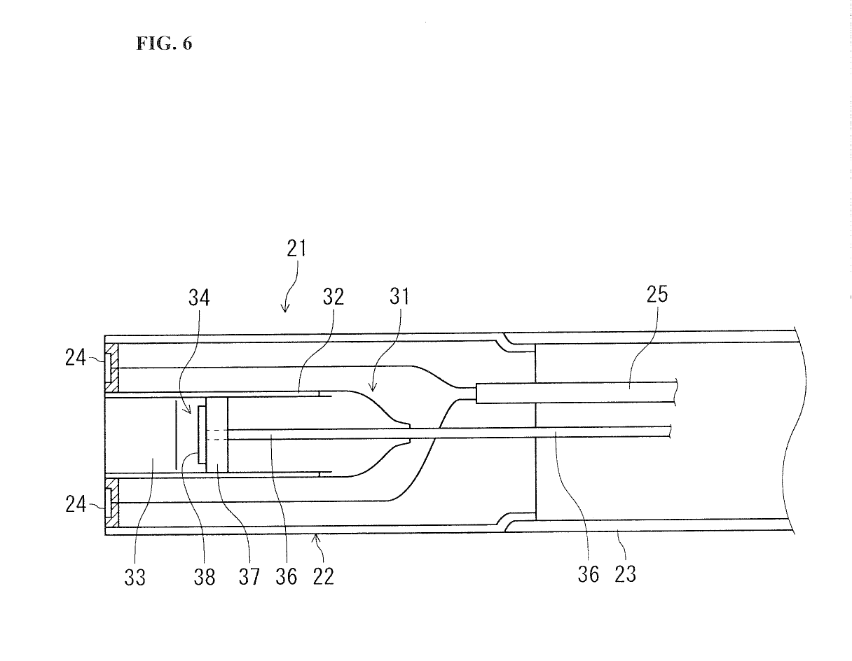

Subsequently, the color camera module 21 will be described in detail, with reference to FIGS. 6 to 11.

As illustrated in FIGS. 5 and 6, the color camera module 21 includes a cylindrical camera head part 22 and the camera unit 31 is arranged inwardly from the distal end surface of the camera head part 22 and an arbitrary number of (for example, eight) light emitting elements (LED: light emitting diodes) 24 (such as those of driving voltage DC 3.3V, for example) are arranged in a circle around the end surface of the camera unit 31. Furthermore, the camera head part 22 is connected to a camera cable 23.

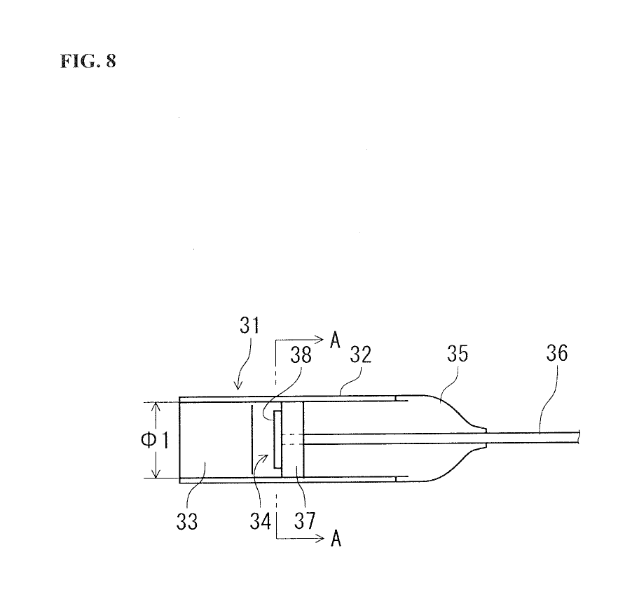

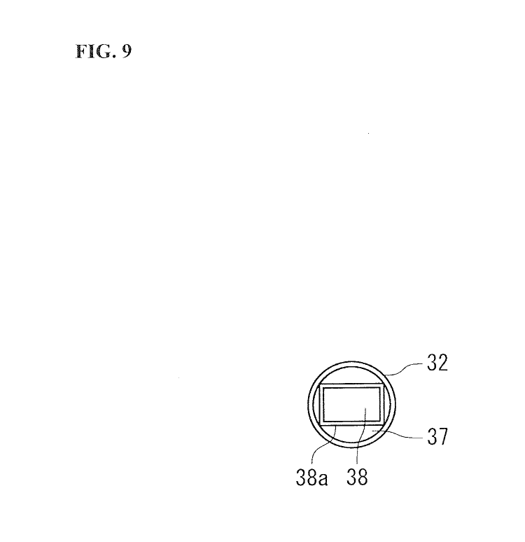

The camera unit 31 will be described in detail below with reference to FIGS. 8 and 9.

The camera unit 31 includes: a cylindrical support cylinder 32, for example, having a diameter of 1.2 mm, an inner diameter of about 1.1 mm, and a length of 3 mm; a condenser lens unit 33 having a diameter Φ1 of 1.1 mm arranged with a light incident end facing one end surface of the support cylinder 32; an image pick-up unit 34 oppositely arranged at a fixed interval apart from the condenser lens unit 33 in the support cylinder 32; a cover member 35 which is fitted in a range from the other end surface side of the support cylinder 32 to the inside of the support cylinder 32; and a signal cable 36 which is connected to the image pick-up unit 34 and led to the rear side through the cover member 35.

The image pick-up unit 34 includes: a disk-shaped support substrate 37 having a diameter of 1.1 mm and firmly fixed with the center aligned with the optical axis of the condenser lens unit 33 in the support cylinder 32; and a color image sensor (CMOS: complementary metal oxide semiconductor) 38 attached in a state where color pixels are arranged in a matrix of 320×240 pixels as the number of pixels on the surface of a sensor substrate 38a having external dimensions of 0.84×0.74 mm and a thickness of 0.1 mm and where the center portion coincides with the optical axis of the condenser lens unit 33, wherein the signal cable 36 is connected to the color image sensor 38 at one end and the other end of the signal cable 36 is led to the rear side through the support substrate 37 and the cover member 35.

The condenser lens unit 33 is, for example, one halving optical characteristics of a viewing angle of 70 degrees and a focus range of 3 to 50 mm.

One end of the light emitting element cable 25 is connected to each of the light emitting elements 24 arranged around the end surface of the camera unit 31. The light emitting element cable 25 is housed in the camera cable 23 together with the signal cable 36 and these cables are led to the dental tube part 12 side.

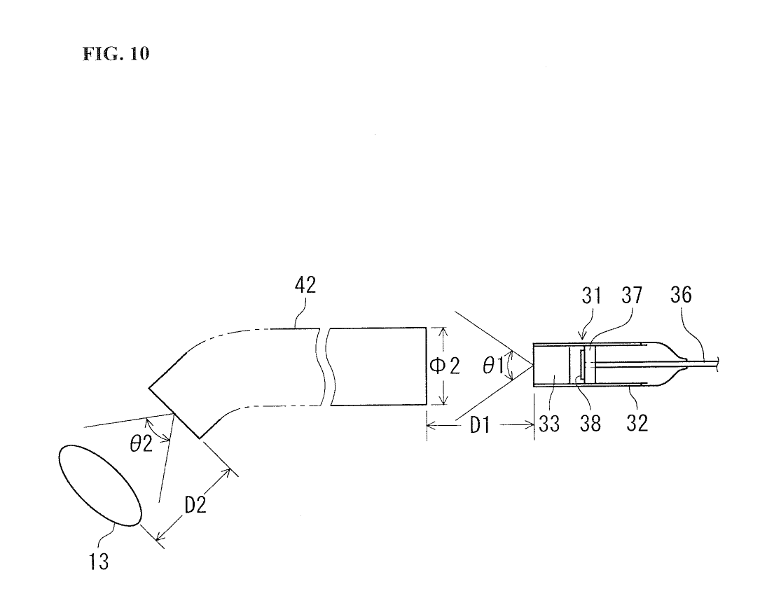

Here, the optical detailed structure of the camera unit 31 included in the color camera module 21, the rod fiber 42, and the objective lens 13 will be described in detail with reference to the enlarged explanatory diagram of FIG. 10.

Regarding the relationship between the light emitting end of the rod fiber 42 and the condenser lens unit 33 of the camera unit 31, a condenser lens unit having a viewing angle θ1 of 70 degrees is used as the condenser lens unit 33, and an interval D1 between the light incident surface of the condenser lens unit 33 and the light emitting end of the rod fiber 42 is set to about 3 mm by employing the light emitting end of the rod fiber 42 having a diameter Φ2 of about 2.4 mm, thereby enabling a beam emitted from the light emitting end of the rod fiber 42 to be received within the range of the viewing angle of the condenser lens unit 33 and thus enabling the light to be guided to the condenser lens unit 33 without hindrance.

On the other hand, regarding the relationship between the objective lens 13 and the light incident end of the rod fiber 42, the light receiving angle θ2 of the rod fiber 42 is about 70 degrees and therefore, for example, a convex lens having a lens diameter on the order of 3 mm and a focal length on the order of 3 mm is used as the objective lens 13 and the interval D2 between the objective lens 13 and the light incident end of the rod fiber 42 is set to about 3 mm., thereby enabling the image pick-up light incident onto the light incident end of the rod fiber 42 via the objective lens 13 to be received within the range of the light receiving angle and thus enabling the light to be guided to the light incident end of the rod fiber 42 without hindrance.

The above configuration provides a built-in camera, hand piece 1 capable of achieving a color image by reliably guiding image pick-up light, entering from the affected area P and incident onto the objective lens 13, to the color image sensor 38, thus being excellent in practical value.

Moreover, the focus range of the condenser lens unit 33 is set to 3 to 50 mm and therefore a clear color image of the affected, area P can be obtained in a wide range when the affected area P is treated by using the built-in camera hand piece 1. Also from this viewpoint, a built-in camera hand, piece 1 excellent in practical value can be provided.

The following describes an example of a wiring processing structure of the light emitting element cable 25 and the signal cable 36 housed in the camera cable 23.

With respect to the light emitting element cable 25 and the signal cable 36, it is possible to employ a configuration in which a contact portion for the light emitting element cable 25 and a contact portion for the signal cable 36 are provided, though not illustrated, between the end surfaces facing each other in the joint portion between the coupling part 11 and the dental tube part 12, and an image signal from the color image sensor 38 sent from the signal cable 36 via the contact portion is taken out to the outside of the built-in camera hand piece 1 and transmitted to the drive control unit 101 described later through the signal output cable 5, which has been introduced into the dental tube part 12, and further light emitting element driving power supplied from the drive control unit 101 is supplied from the light emitting element driving cable 6, which has been introduced into the dental tube part 12, to the light emitting element cable 25 via the contact portion.

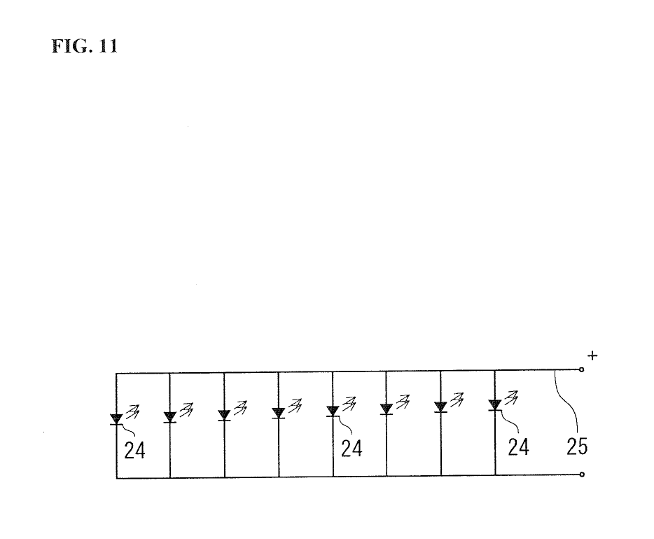

FIG. 11 illustrates a driving circuit of the light emitting element 24. For example, eight light emitting elements 24 are connected in parallel and DC voltage supplied from the light emitting element power supply unit 103 via the light emitting element driving cable 6 and the light emitting element cable 25 is applied to an area between the anode and the cathode of each light emitting element 24 to drive the light emitting elements 24 to illuminate.

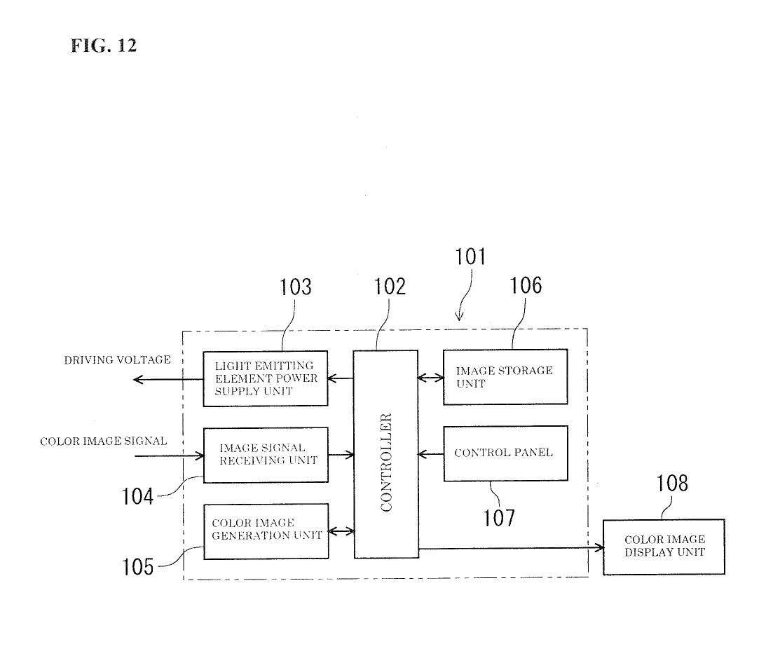

The following describes the schematic configuration of the drive control unit 101 for driving the built-in camera hand piece 1 of the first embodiment, with reference to FIG. 12.

The drive control unit 101 includes: a controller 102 for controlling the operation of the entire built-in camera hand piece 1; a light emitting element power supply unit 103 for supplying each light emitting element 24 with driving voltage (for example, DC 3.3V); an image signal receiving unit 104 for receiving an image signal from the camera unit 31; a color image generation unit 105 for generating a color image of a row of teeth or the like in the oral cavity on the basis of the received image signal; an image storage unit 106 for storing the generated color image; and a control panel 107 equipped with various operation buttons necessary for operations of the built-in camera hand piece 1.

Moreover, the drive control unit 101 of the first embodiment picks up an image by using the camera unit 31, generates a color image by using the color image generation unit 105, and sends the generated color image to the color image display unit 108 composed of a color liquid crystal display and the like to display the color image on a screen.

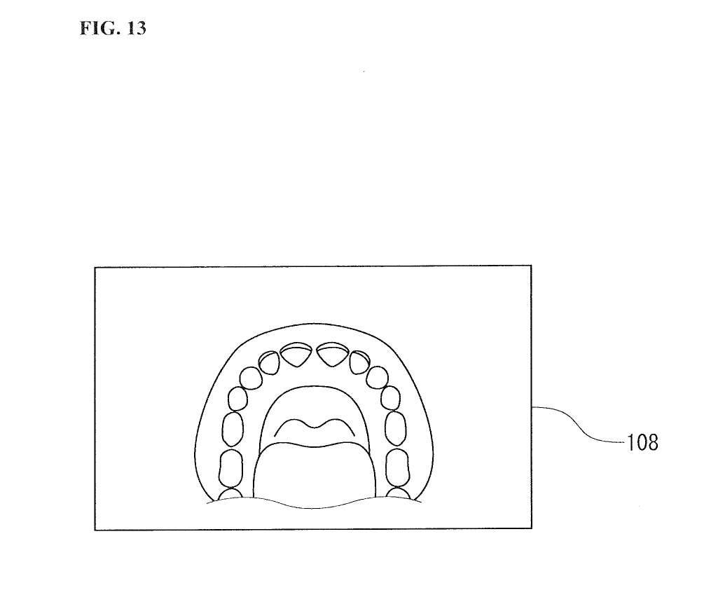

Thereby, as illustrated in FIG. 13, a practitioner is able to check even a portion of the distal wall of a row of teeth in the oral cavity, which is difficult to be seen with the practitioner's naked eye, on the color image.

According to the built-in camera hand piece 1 of the first embodiment, an air flow from the defogging air flow path mechanism 4 prevents the adhesion of cutting waste or water droplets generated during a cutting work in the affected area P to the outer surface of the image pick-up window 15 and the air flow blows away the adhering cutting waste or water droplets to defog the image pick-up window 15, by which the image pick-up light from the affected area P is incident onto the objective lens 13 in a clear state and is guided to the color image sensor 38, thereby enabling a clear color image to be picked up.

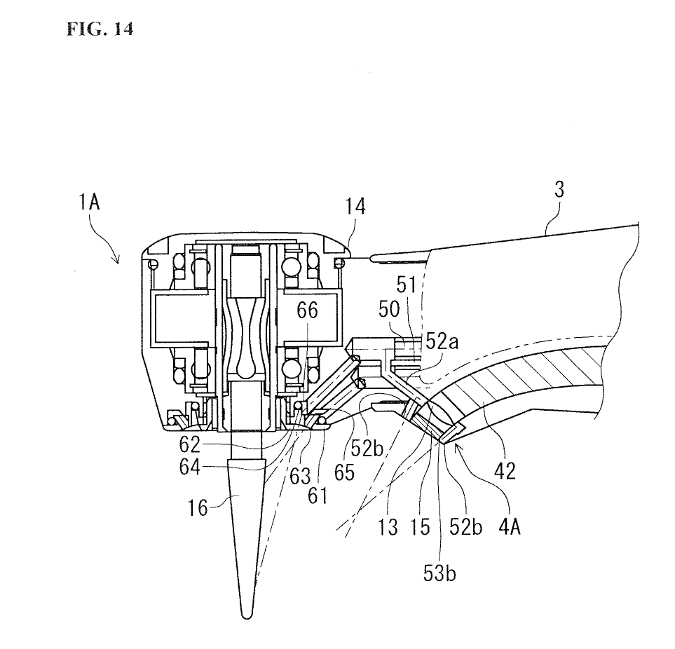

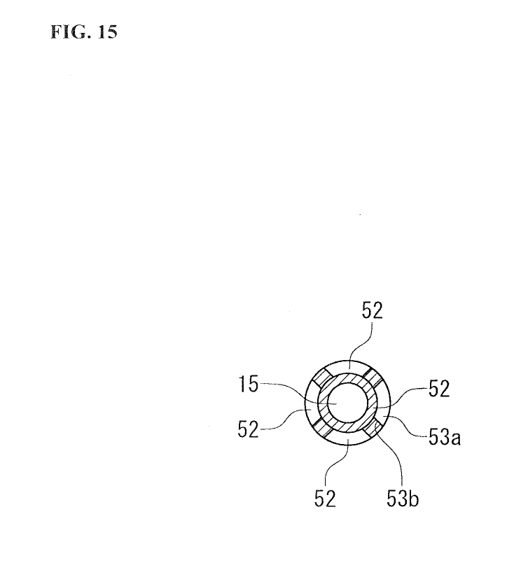

Subsequently, a built-in camera hand piece 1A according to a variation of the built-in camera, hand piece 1 of the first embodiment will be described with reference to FIGS. 14 and 15.

Although the basic configuration of the built-in camera hand piece 1A according to the variation is the same as the basic configuration of the built-in camera hand piece 1 of the first embodiment, the built-in camera hand piece 1A is characterized by that a defogging air flow path mechanism 4A for defogging the outer surface of the image pick-up window 15 is added around the image pick-up window 15 in substantially the same manner as in the case illustrated in FIG. 3.

Specifically, there is provided a defogging air flow path 52a having the same structure as in the case illustrated in FIG. 4, such that the defogging air flow path 52a is branched on the way from the air flow path 50, and having a structure of surrounding the outer surface portion of the image pick-up window 15, so that an air flow is injected in a conical form in the forward direction of the image pick-up window 15 from an opening 52b of the defogging air flow path 52a.

FIG. 15 is a diagram, of the image pick-up window 15 viewed from a direction opposite to the image pick-up window 15. A cylindrical camera head part 22 is inserted and arranged into the central portion of a pipe 53a which forms an air flow path communicating with the defogging air flow path 52a with a guide cylinder 53b as a guide and a clearance generated between the pipe 53a and the guide cylinder 53b forms the opening 52b around the image pick-up window 15. The air flow is injected in a conical form from the opening 52b.

The opening 52b may be a continuous groove surrounding the image pick-up window 15 or may be a chain line-like groove. Further, the opening 52b may be perforations radially arranged around the image pick-up window 15.

According to the defogging air flow path mechanism 4A illustrated in FIG. 14, due to the effect of an air curtain generated by the air flow injected in a conical form from the opening 52b of the defogging air flow path 52a, the cutting waste or water droplets generated during a cutting work in the affected area P can be prevented from adhering to the outer surface of the image pick-up window 15, by which the image pick-up light from the affected area P is incident onto the objective lens 13 in a clear state while defogging the image pick-up window 15 and is guided to the color image sensor 38, thereby enabling a good-quality color image to be picked up.

This configuration enables a good field of vision to be secured in the image pick-up window 15, while guiding the image pick-up light from the affected area P in a clear state to the color image sensor 38 of the camera unit 31 through the objective lens 13 and the rod fiber 42, picking up a clear color image, and displaying the image on a screen by using the color image display unit 108.

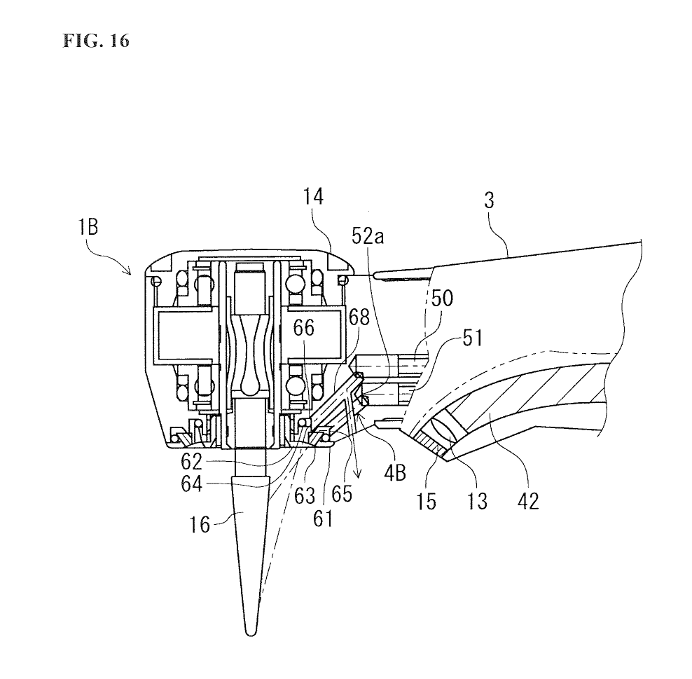

Subsequently, a built-in camera hand piece 1B according to another variation of the built-in camera hand piece 1 according to the first embodiment will be described with reference to FIG. 16.

Although the basic configuration of the built-in camera hand piece 1B according to another variation illustrated in FIG. 16 is substantially the same as the basic configuration of the built-in camera hand piece 1 illustrated in FIG. 3, the built-in camera hand piece 1B is characterized by that a defogging air flow path mechanism 4B having an air curtain structure in a straight form is provided, instead of the defogging air flow path mechanism 4.

The defogging air flow path mechanism 4B has a configuration in which, as illustrated in FIG. 16, a defogging air flow path 52a, similar to that in FIG. 4, provided in the inside of the head part 14 has a straight structure without being directed to the image pick-up window 15, so that an air flow is injected from the defogging air-flow path 52a toward the front region of the outer surface of the image pick-up window 15.

According to the defogging air flow path mechanism 4B, an air flow is injected from the defogging air flow path 52a toward the front region of the outer surface of the image pick-up window 15 to form an air curtain, thereby providing the same advantageous effects as those of the defogging air flow path mechanism 4 illustrated in FIG. 4 or the defogging air flow path mechanism 4A illustrated in FIG. 14.

### Second Embodiment

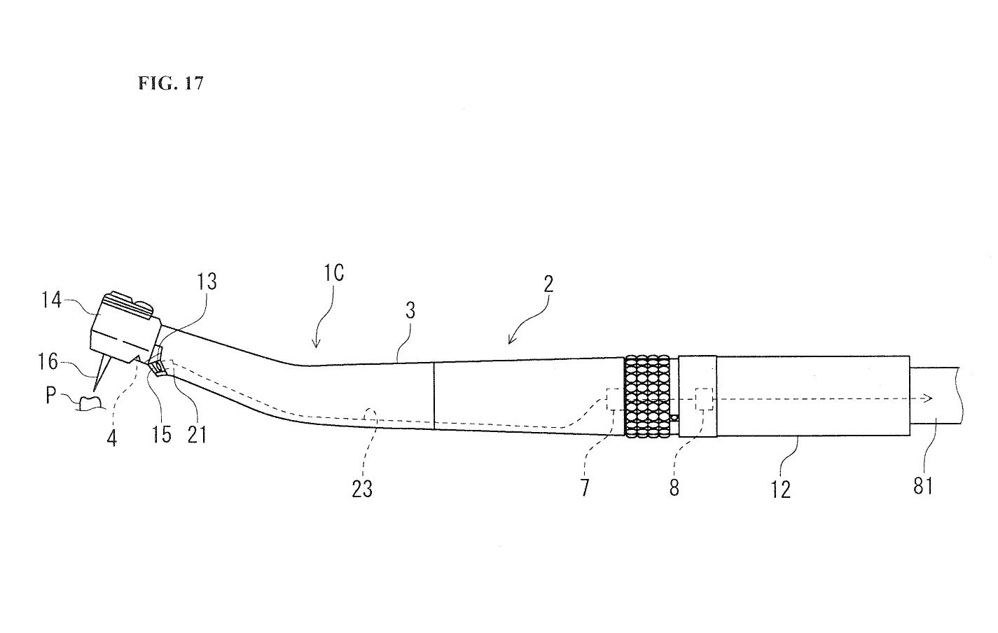

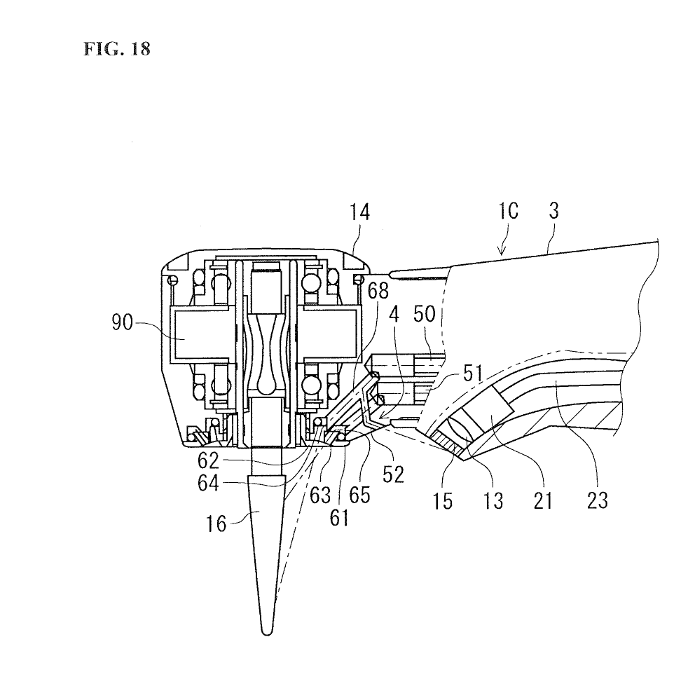

Subsequently, a built-in camera hand piece 1C according to a second embodiment of the present invention will be described with reference to FIGS. 17 and 18.

Although the entire configuration of the built-in camera hand piece 1C according to the second embodiment is substantially the same as that of the built-in camera hand piece 1 of the first embodiment and the defogging air flow path mechanism 4 is the same as in the first embodiment, the built-in camera hand, piece 1C is characterized by that the rod fiber 42 is omitted and the color camera module 21 is arranged in the inside of the grip part 3 and in the inside of the objective lens 13 as schematically illustrated in FIGS. 17 and 18.

Further, although a detailed wiring structure is omitted, the built-in camera hand piece 1C is characterized by that a camera cable 23 connected to the color camera module 21 is guided into the dental tube part 12 passing through the inside of the grip part 3 and of the coupling part 11 and via connectors 7 and 8 and that the signal cable 36 and the light emitting element cable 25 mounted inside the camera cable 23 are connected to the signal output cable 5 and the light emitting element driving cable 6 included in the electric cable 84.

Also according to the built-in camera hand piece 1C of the second embodiment, with the configuration in which the color camera module 21 is arranged inside the grip part 3 and near the head part 14, an air flow from the defogging air flow path mechanism 4 prevents the adhesion of cutting waste or water droplets generated during a cutting work in the affected area P to the outer surface of the image pick-up window 15 to defog the image pick-up window 15 in the same manner as in the built-in camera hand piece 1 according to the first embodiment, by which the image pick-up light from the affected area P is incident onto the objective lens 13 in a clear state and is guided to the color image sensor 38, thereby enabling a clear color image to be picked up.

Also in the built-in camera hand piece 1C according to the second embodiment, it is naturally possible to use the defogging air flow path mechanism 4A as illustrated in FIG. 14 or the defogging air flow path mechanism 4B as illustrated in FIG. 16 and to provide the same working effect as in the cases described above.

### Third Embodiment

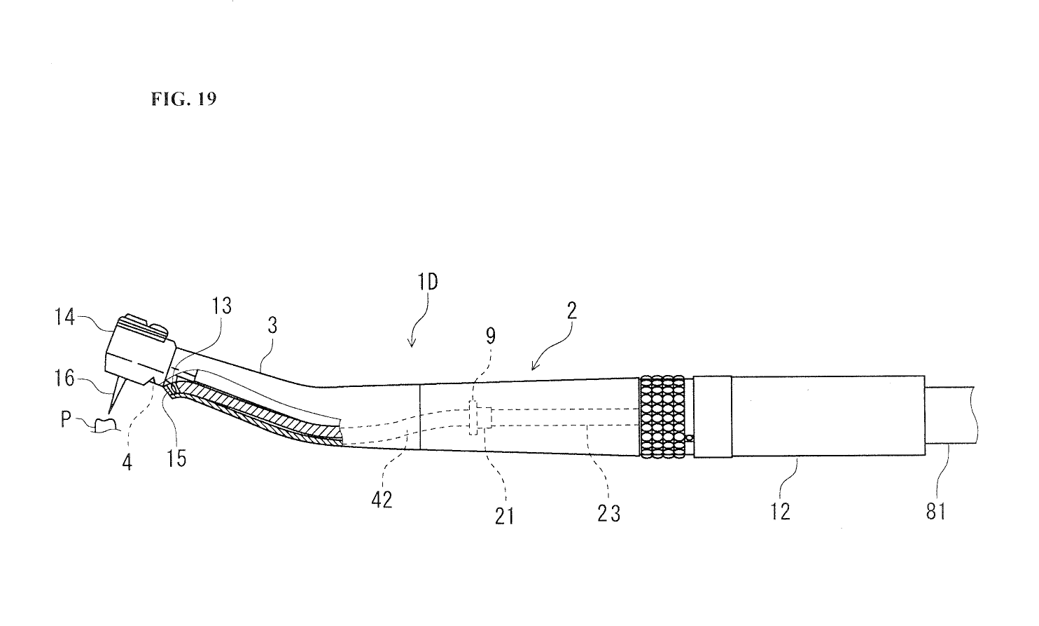

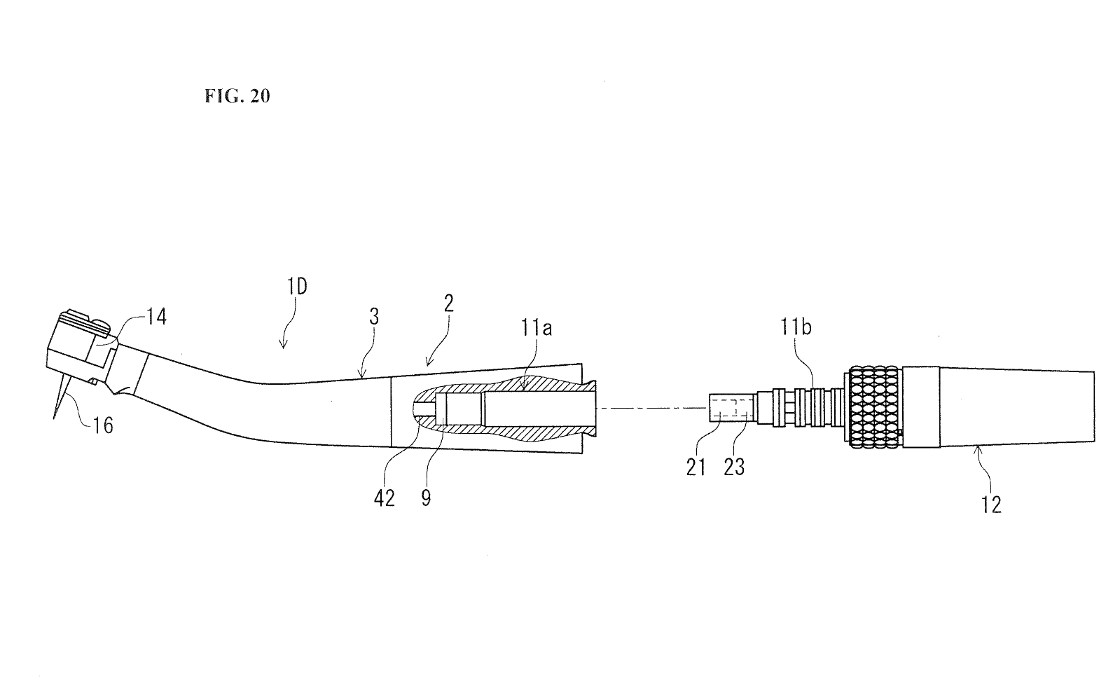

Subsequently, a built-in camera hand piece 1D according to a third embodiment of the present invention will be described with reference to FIGS. 19 and 20.

Although the entire configuration of the built-in camera hand piece 1D according to the third embodiment is substantially the same as the entire configuration of the built-in camera hand piece 1 according to the first embodiment and the defogging air flow path mechanism 4 is the same as in the first embodiment, the built-in camera hand piece 1B is characterized by that, as schematically illustrated in FIGS. 19 and 20, the color camera module 21 is arranged in a distal end portion present inside the coupling part 11b in the dental tube part 12 and that the rod fiber 42 is arranged with the incident end thereof facing the objective lens 13 and with the emitting end of the rod fiber 42 facing the color camera module 21 at a predetermined interval apart therefrom via an optical coupling 9.

Also in this case, the signal cable 36 and the light emitting element cable 25 mounted inside the camera cable 23 in the color camera module 21 are connected to the signal output cable 5 and the light emitting element driving cable 6 in the electric cable 84, though the detailed wiring structure is omitted.

Also according to the built-in camera hand piece 1D of the third embodiment, with the configuration in which the color camera module 21 is arranged in the distal end portion of the coupling part 11b in the dental tube part 12, an air flow from the defogging air flow path mechanism 4 prevents the adhesion of cutting waste or water droplets generated during a cutting work in the affected area P to the outer surface of the image pick-up window 15 to defog the image pick-up window 15 in the same manner as in the built-in camera hand piece 1 according to the first embodiment, by which the image pick-up light from the affected area P is incident onto the objective lens 13 in a clear state and is guided to the color image sensor 38, thereby enabling a clear color image to be picked up.

Also in the built-in camera hand piece 1D according to the third embodiment, it is naturally possible to use the defogging air flow path mechanism 4A as illustrated, in FIG. 14 or the defogging air flow path mechanism 4B as illustrated in FIG. 16 and to provide the same working effect as in the cases described above.

Although the air turbine hand piece has been described as in the above in the preferred embodiments of the present invention by giving examples, the present invention is not limited to a hand piece in which the cutting tool for treating an affected area is driven by a rotational force of the air turbine, but the cutting tool may be driven by a rotational force of an electric-drive micromotor or a rotational force of an air motor. Moreover, the cutting tool for treating the affected area may be a cutting tool with ultrasonic vibration such as an ultrasonic scaler, and the present invention is also applicable to these hand pieces.

Furthermore, the present invention is not limited to the above embodiments, and constituent elements can be modified and embedded in the stage of practice without departing from the spirit and scope of the invention.

### DESCRIPTION OF REFERENCE NUMERALS

1 Image pick-up window defogging function-equipped built-in camera hand piece

1A Image pick-up window defogging function-equipped built-in camera hand piece

1B Image pick-up window defogging function-equipped built-in camera hand, piece

1C Image pick-up window defogging function-equipped built-in camera hand piece

1D Image pick-up window defogging function-equipped built-in camera hand piece

2 Hand piece body

3 Grip part

4 Defogging air flow path mechanism

4A Defogging air flow path mechanism

4B Defogging air flow path mechanism

5 Signal output cable

6 Light emitting element driving cable

7 Connector

8 Connector

9 Optical coupling

11 Coupling part

11a Coupling part (on the hand piece body side)

11b Coupling part (on the dental tube part side)

12 Dental tube part

13 Objective lens

14 Head part

15 Image pick-up window

16 Cutting tool

17 Connector

18 Connector

21 Color camera module

22 Camera head part

23 Camera cable

24 Light emitting element

25 Light emitting element cable

31 Camera unit

32 Support cylinder

33 Condenser lens unit

34 Image pick-up unit

35 Cover member

36 Signal cable

37 Support substrate

38 Color image sensor

38a Sensor substrate

41 Image pick-up optical system

42 Rod fiber

50 Air flow path

51 Water flow path

52 Defogging air flow path

52a Befogging air flow path

52b Opening

53a Pipe part

53b Guide cylinder

61 Ring

62 Nut

63 Water injection hole

64 Air injection hole

65 Peripheral groove

66 Peripheral groove

67 Connecting pipeline

68 Connecting pipeline

81 Connection hose

82 Air pipe

83 Water pipe

84 Electric cable

90 Air turbine

91 Air flow path system

92 Water flow path system

101 Drive control unit

102 Controller

103 Light emitting element power supply unit

104 Image signal receiving unit

105 Color image generation unit

106 Image storage unit

107 Control panel

108 Color image display unit

P Affected area

1. An image pick-up window defogging function-equipped built-in camera hand piece comprising:
a hand piece body equipped with a head part on the distal end side with a cutting tool for treating an affected area detachably mounted on the head part;
an air flow path, system for guiding compressed air to the head part;
a water flow path system for guiding pressurized water for a water flow injected toward the cutting tool;
an image pick-up window for the camera provided in a surrounding region of a position where the cutting tool on the head part is mounted;
a built-in camera for picking up affected area image information incident through the image pick-up window; and
an image pick-up optical system arranged in a range from the inside of the image pick-up window to the built-in camera,
wherein a defogging air flow path mechanism is provided in a surrounding position of the image pick-up window, the defogging air flow path mechanism branching from the air flow path system and injecting an air flow within a region from the outer surface of the image pick-up window to a cutting site of the cutting tool to shield an area between the outer surface of the image pick-up window and the cutting site by the air flow to defog the outer surface of the image pick-up window.

2. An image pick-up window defogging function-equipped built-in camera hand piece comprising:
a hand piece body having a grip part equipped with a head part on the distal end side with a cutting tool for treating an affected area detachably mounted on the head part and having one coupling part at the rear end of the grip part;
a dental tube part having the other coupling part detachably mounted on the hand piece body;
an air flow path system tor guiding compressed air for an air flow injected from the head part through the dental tube part toward the cutting tool;
a water flow path system, for guiding pressurized water for a water flow injected from the head part toward the cutting tool, the pressurized water being pressure-fed to the head part of the hand piece body through the dental tube part;
an image pick-up window for the camera provided in a surrounding region of a position where the cutting tool on the head part is mounted;
a built-in camera for picking up affected area image information incident through the image pick-up window; and
an image pick-up optical system arranged in a range from the inside of the image pick-up window to the built-in camera.
wherein a defogging air flow path mechanism is provided in a surrounding position of the image pick-up window, the defogging air flow path mechanism branching from the air flow path system arid injecting an air flow within a region from the outer surface of the image pick-up window to a cutting site of the cutting tool to shield an area between the outer surface of the image pick-up window and the cutting site by the air flow to defog the outer surface of the image pick-up window.

3. The image pick-up window defogging function-equipped built-in camera hand piece according to claim 1, wherein the hand piece body is an air turbine rotationally driven by a rotational force of an air flow and wherein the air flow path system is for use in guiding both of an air flow pressure-fed to the air turbine and an air flow injected from the head part toward the cutting tool.

4. The image pick-up window defogging function-equipped built-in camera hand piece according to claim 1, wherein the built-in camera is provided in the distal end portion of the coupling part located inside the hand piece body.

5. The image pick-up window defogging function-equipped built-in camera hand piece according to claim 1, wherein the built-in camera is provided inside the image pick-up window.

6. The image pick-up window defogging function-equipped built-in camera hand piece according to claim 1, wherein the built-in camera is provided in the distal end portion of the coupling part of the dental tube part.

7. The image pick-up window defogging function-equipped built-in camera hand piece according to claim 1, wherein the defogging air flow path mechanism blows an air flow from a defogging air flow path branching from the air flow path system toward the outer surface of the image pick-up window to defog the image pick-up window.

8. The image pick-up window defogging function-equipped built-in camera hand piece according to claim 1, wherein the defogging air flow path mechanism injects an air flow from a defogging air flow path brandling from the air flow path system and reaching a position around the outer surface portion of the image pick-up window, in a conical form in the forward direction of the image pick-up window to form an air curtain to defog the image pick-up window.

9. The image pick-up window defogging function-equipped built-in camera hand piece according to claim 1, wherein the defogging air flow path mechanism injects an air flow straight from a defogging air flow path branching from the air flow path system toward a space region between the outer surface of the image pick-up window and the cutting tool to form an air curtain to defog the image pick-up window.

10. The image pick-up window defogging function-equipped built-in camera hand piece according to claim 2, wherein the built-in camera is provided in the distal end portion of the coupling part located inside the hand piece body.

11. The image pick-up window defogging function-equipped built-in camera hand piece according to claim 2, wherein the built-in camera is provided inside the image pick-up window.

12. The image pick-up window defogging function-equipped built-in camera hand piece according to claim 3, wherein the built-in camera is provided inside the image pick-up window.

13. The image pick-up window defogging function-equipped built-in camera hand piece according to claim 2, wherein the built-in camera is provided in the distal end portion of the coupling part of the dental tube part.

14. The image pick-up window defogging function-equipped built-in camera hand piece according to claim 2, wherein the defogging air flow path mechanism blows an air flow from a defogging air flow path branching from the air flow path system toward the outer surface of the image pick-up window to defog the image pick-up window.

15. The image pick-up window defogging function-equipped built-in camera hand piece according to claim 2, wherein the defogging air flow path mechanism injects an air flow from a defogging air flow path branching from the air flow path system and reaching a position around the outer surface portion of the image pick-up window, in a conical form in the forward direction of the image pick-up window to form an air curtain to defog the image pick-up window.

16. The image pick-up window defogging function-equipped built-in camera hand piece according to claim 2, wherein the defogging air flow path mechanism injects an air flow straight from a defogging air flow path branching from the air flow path system toward a space region between the outer surface of the image pick-up window and the cutting tool to form an air curtain to defog the image pick-up window.
